(19) United States
(12) Patent Application Publication
(10) Pub. No.: US 2012/0116218 A1
(43) Pub. Date: May 10, 2012

(54) METHOD AND SYSTEM FOR DISPLAYING ULTRASOUND DATA

(76) Inventors: Jennifer Martin, North Prairie, WI (US); Gary Cheng How Ng, Bothell, WA (US)

(21) Appl. No.: 12/943,572

(22) Filed: Nov. 10, 2010

(51) Int. Cl. A61B 8/00

(52) U.S. Cl. 600/437

(57) ABSTRACT

Methods and systems for displaying ultrasound data are provided. One method includes acquiring ultrasound image data and physiological monitoring data during an ultrasound imaging scan, generating quantitative ultrasound data from the acquired ultrasound image data and correlating the quantitative ultrasound data with the physiological monitoring data. The method also includes displaying the correlated quantitative ultrasound data and physiological monitoring data time aligned on a display.

### BACKGROUND OF THE INVENTION

The subject matter disclosed herein relates generally to methods and systems for displaying ultrasound data, and more particularly to displaying quantitative ultrasound data correlated with physiological monitoring data.

Diagnostic medical imaging systems typically include a scan portion and a control portion having a display. For example, ultrasound imaging systems usually include ultrasound scanning devices, such as ultrasound probes having transducers that are connected to an ultrasound system to control the acquisition of ultrasound data by performing various ultrasound scans (e.g., imaging a volume or body). The ultrasound systems are controllable to operate in different modes of operation and to perform different scans. The acquired ultrasound data then may be displayed, which may include images of a region of interest.

Both physical exams (e.g., joint pain assessment) and ultrasound imaging (e.g., color flow ultrasound imaging) can be used to assess different medical conditions and the success of treatment of those conditions, such as long term treatment. For example, using ultrasound imaging, color flow ultrasound data can be used to assess the degree of inflammation in joints for rheumatoid arthritis or the degree of angiogenesis in tumors. The amount of color displayed within a region of interest (ROI) can be trended over subsequent exams of the same patient to assess the progression of a treatment. However, the measurement of the amount of color (imaged blood flow) can be highly variable because of environmental conditions. For example, a hot day or a hot room results in more flow in the joints. Additionally, in some studies, the desired data may be acquired when a physiological parameter is in different states.

Thus, long term treatment assessment using quantitative ultrasound data may be difficult to perform because of varying conditions, particularly varying environmental or exam conditions.

### BRIEF DESCRIPTION OF THE INVENTION

In accordance with various embodiments, a method for displaying ultrasound data is provided. The method includes acquiring ultrasound image data and physiological monitoring data during an ultrasound imaging scan, generating quantitative ultrasound data from the acquired ultrasound image data and correlating the quantitative ultrasound data with the physiological monitoring data. The method also includes displaying the correlated quantitative ultrasound data and physiological monitoring data time aligned on a display.

In accordance with other various embodiments, an ultrasound display is provided that includes an ultrasound image corresponding to one frame in an acquired ultrasound data image loop, a quantitative display portion having a time aligned graph and at least one plot of quantitative ultrasound data on the time aligned graph. The ultrasound display also includes at least one physiological monitoring trace on the time aligned graph.

In accordance with yet other various embodiments, an ultrasound system is provided that includes a probe configured to acquire ultrasound image data, a physiological monitoring device configured to acquire physiological monitoring data corresponding to the acquired ultrasound image data and a processor configured to correlate the acquired ultrasound image data and the acquired physiological monitoring data. The ultrasound system also includes a display configured to display quantitative ultrasound data based on the ultrasound image data and the physiological monitoring data time aligned.

### BRIEF DESCRIPTION OF THE DRAWINGS

FIG. 1 is a block diagram illustrating a process for generating and displaying quantitative ultrasound data in combination with monitoring data in accordance with various embodiments.

FIG. 2 is a flowchart of a method to acquire and display correlated quantitative ultrasound data and patient monitoring data in accordance with various embodiments.

FIG. 3 is an exemplary display illustrating quantitative ultrasound data displayed in combination with monitoring data in accordance with various embodiments.

FIG. 4 is a simplified block diagram of an ultrasound system in which various embodiments may be implemented.

FIG. 5 is a detailed block diagram of the ultrasound system of FIG. 4.

FIG. 6 is a block diagram of an ultrasound processor module of the ultrasound system of FIG. 5 formed in accordance with various embodiments.

FIG. 7 is a diagram illustrating a three-dimensional (3D) capable miniaturized ultrasound system in which various embodiments may be implemented.

FIG. 8 is a diagram illustrating a 3D capable hand carried or pocket-sized ultrasound imaging system in which various embodiments may be implemented.

FIG. 9 is a diagram illustrating a 3D capable console type ultrasound imaging system in which various embodiments may be implemented.

### DETAILED DESCRIPTION OF THE INVENTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Various embodiments provide a system and method for associating or correlating physiological monitoring data to quantitative ultrasound data, for example, extracted from an ultrasound data loop (also referred to as a cine loop). At least one technical effect of the various embodiments is the reduction in the variance in treatment monitoring results. Additionally, by practicing various embodiments, external variables (e.g., different environmental conditions) may be monitored and potentially corrected when acquiring quantification data. For example, one or more data points may be adjusted or excluded based on the state of a patient.

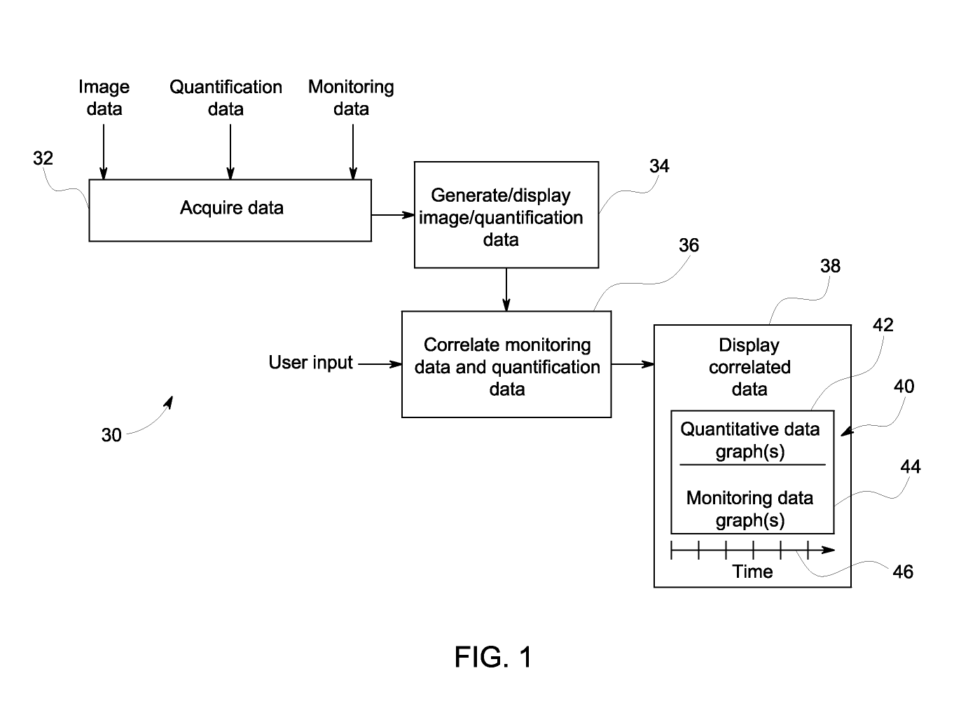

One embodiment of a process 30 for generating and displaying ultrasound data, and in particular, quantitative ultrasound data, in combination with monitoring data, is illustrated in FIG. 1. The process 30 includes acquiring data at 32, which may include image data, quantification data and/or monitoring data, among other data. For example, an ultrasound probe is used to acquire image data and quantification data (e.g., fraction of a region of interest (ROI) that includes color pixels corresponding to blood flow generated from the image data) and a monitoring device, such as an electrocardiography (ECG) device, is used to acquire physiological data. It should be noted that physiological data other than or in addition to ECG data may be acquired. For example, other physiological data, such as pulse-oximetry, temperature, and blood pressure may also or alternatively be acquired.

As used herein, quantitative ultrasound data refers to any quantifiable, plottable, measurable, determinable or other numerical data acquired, determined and/or generated from ultrasound data, which may be obtained using different types of ultrasound data acquisition.

Using the data acquired at 32, image and/or quantification data is generated and displayed at 34. For example, a graph of quantification data as a function of time is displayed for one or more ROIs (which also may be displayed). The monitoring data and quantification data are correlated at 36, for example, such that the monitoring data is plotted on the same graph and time scale as the quantification data. One or more user inputs also may be received, for example, to scale the quantification graph and results with the monitoring input data values. As another example, the received user inputs may identify portions or the graph (and accordingly the results) to exclude or include based upon the monitoring input values, such as the patient state based on measured physiological values. It should be noted that in some embodiments, acquisition of image frames as part of the data acquired at 32 may be triggered only if the monitoring input value, for example, a physiological input value exceed a threshold, such as a predetermined threshold.

It also should be noted that correlation as used herein refers to any type of association of data and is not limited, for example, to a mathematical correlation. Accordingly, in various embodiments, the correlation of the monitoring data and quantification data includes, for example, time associating the data such that the physiological value (e.g., at 1.2 seconds after acquisition start) is associated with the quantitative ultrasound value at the frame acquired at that time.

The correlated data, for example, the graph of the quantification data and monitoring data plotted on the same graph and time scale is then displayed or updated (e.g., based on a user input) at 38. For example, one or more quantification data plots or curves corresponding to a ratio or fraction of color pixels (corresponding to blood flow) may be displayed in a time aligned manner with physiological data, such as ECG or heart rate data. As shown in FIG. 1, a quantitative display 40 may be provided that may form part of an ultrasound display having other information displayed thereon. The quantitative display 40 includes one or more quantitative data graphs/plots 42 and one or more monitoring data graphs/plots 44 aligned in time (represented by the time scale 46). Thus, at each point in time during acquisition of the ultrasound data used to generate the quantitative data, corresponding monitoring data, such as physiological monitoring data is shown, allowing a user to determine the value(s) of the physiological monitoring data at the different points in time.

Thus, in operation, various embodiments acquire physiological signals using an ultrasound system, which may be used, for example, to gate, reject or scale color quantification data. For example, ECG data used for gating and triggering acquisition of ultrasound data may be correlated and displayed with the color quantification data.

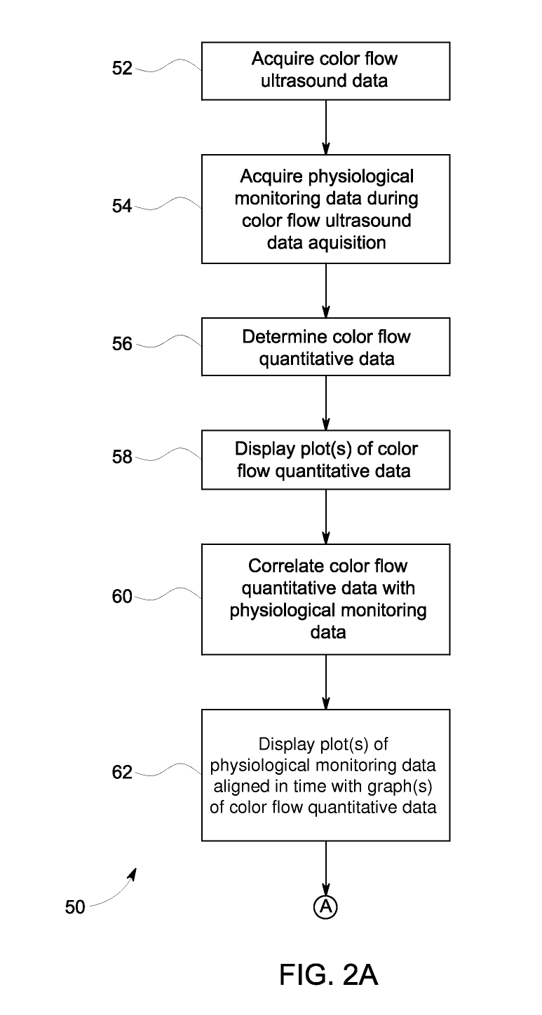

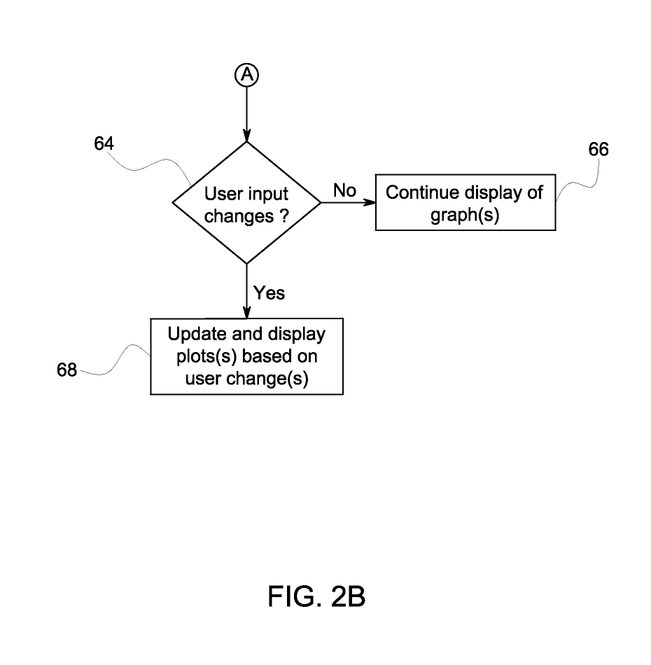

Various embodiments may include a method 50 as illustrated in FIG. 2 to acquire and display correlated quantitative ultrasound data and monitoring data, such as patient monitoring data. It should be noted that although the method 50 is described in connection with generating color flow ultrasound data and correlating the data with particular physiological data, the method 50 is not limited to particular quantitative ultrasound data or physiological data.

The method 50 includes acquiring ultrasound data, and in particular, acquiring color flow ultrasound data at 52. The ultrasound data acquired at 52 may be acquired using any suitable method and ultrasound system. In general, color flow ultrasound data includes data that may be used to produce a color-coded map of Doppler shifts superimposed onto a B-mode ultrasound image (color flow maps). In operation, color flow imaging uses pulses along each of a plurality of color scan lines of the image to obtain a mean frequency shift and a variance at each area of measurement. This frequency shift is displayed as a color pixel. The imaging system then repeats the process for multiple lines to form the color image, which is superimposed onto the B-mode image. It should be noted that the transducer elements are switched rapidly between B-mode and color flow imaging to give the appearance of a combined simultaneous image.

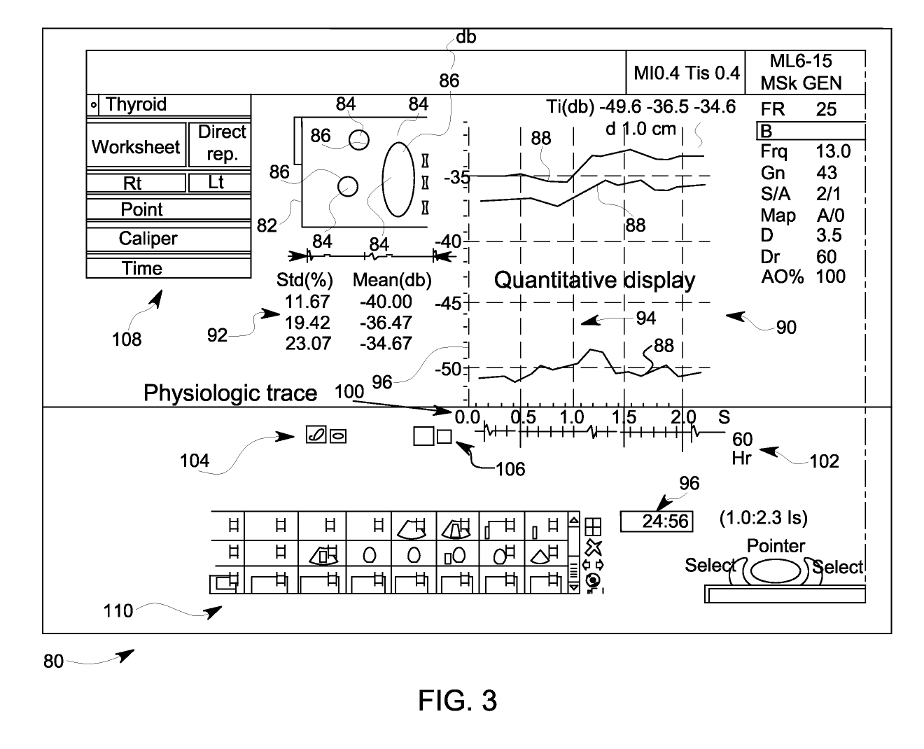

In various embodiments, the assignment of color to frequency shifts is based on direction, for example, red for Doppler shifts towards the ultrasound beam and blue for Doppler shifts away from the ultrasound beam, with magnitude shown using different color hues or lighter saturation for higher frequency shifts. Thus, for example, as shown in FIG. 3, illustrating an exemplary display 80 (e.g., a user interface) a color flow ultrasound image 82 may be displayed having a color-coded map of Doppler shifts (illustrated by colored regions 84, such as different shades of red, blue, etc.) superimposed onto a B-mode ultrasound image. At least some of the colored regions 84 are encompassed within user selected regions of interest (ROIs) 86, which are identified by the outlined regions that may be user traces from a user input device (e.g., a mouse). Thus, the color flow data used to generate the ultrasound image 82 includes acquired Doppler and B-mode ultrasound data.

Referring again to FIG. 2, physiological monitoring data is also acquired at 54 during acquisition of the color flow ultrasound data. The physiological monitoring data may be any type of physiological data, for example, of a patient that is acquired during ultrasound data acquisition (e.g., image data acquisition). The physiological monitoring data may include, for example, ECG, heart rate, pulse-oximetry, temperature, blood pressure and/or breathing data, among other data. It should be noted that any suitable monitoring device may be used to acquire the physiological monitoring data and be separate from or included as part of the ultrasound system. For example, the ultrasound system may include an input for connecting to and receiving physiological data signals from the physiological monitoring device.

Once the ultrasound data and/or physiological data has been acquired, or as the data is being acquired, quantitative data is determined, which in this embodiment includes determining color flow quantitative data at 56. For example, the quantitative data may include color blood flow data wherein a determination is made as to an amount or ratio of blood flow through an ROI based on a number of color flow pixels in the ultrasound image data indicating varying levels of blood flow. For example a blood flow ratio for one or more ROIs may be determined as follows: (number of color flow pixels/total number of pixels) in the ROIs.

The acquired quantitative data, which in various embodiments includes calculated values (e.g., the blood flow ratio), are displayed at 58. For example, one or more quantitative plots 88 as illustrated in FIG. 3 may be displayed in a quantitative display portion 90 of the display 80. In the illustrated embodiment, the plots 88 are curves of the mean B-mode values across all frames in a loop (e.g., a 2 second loop), which is defined by a time period. Thus, the plots 88 correspond to mean B-mode values over time acquired by the ultrasound system. It should be noted that different values or quantitative data may be plotted, for example, based on the type of examination, etc. Additionally, each plot 88 corresponds to one of the ROIs 86, such that the plot 88 corresponds to quantitative data within that ROI 86. The plots 88 may be color coded based on the color of the outlines of the ROIs 86 to allow association and easier visualization of the plots 88 with the ROIs 86.

Additionally, numerical quantitative data 92 calculated from the acquired ultrasound data may also be displayed. For example, a standard deviation for the B-mode data and a mean value for the B-mode data in each ROI for a particular frame of ultrasound data is illustrated. It should be noted that different types of numerical quantitative data 92 may be displayed, such as the blood flow ratio as described herein. Additionally, the frame of ultrasound data corresponding to the displayed ultrasound image 82 and the numerical quantitative data 92 is identified by a line 94 on the graph 96 in the quantitative display portion 90. The line 94 may automatically move during display of the cine loop over time or may be manually moved and stopped by a user at a particular frame. It should be noted that frame data 98 may be displayed indicating the total number of acquired frames of data. For example, in the illustrated embodiment, 24:56 means that the current displayed image is from frame 24 of a total of 56 frames.

Referring again to FIG. 2, the quantitative data is correlated with the physiological monitoring data at 60. It should be noted that the steps of the method 50 may be performed in any order and are not limited to the order shown. Additionally, the two or more of the steps may be performed concurrently or simultaneously. Correlating the quantitative data and the physiological data in some embodiments includes determining for each point in time within a defined time period (e.g., cine loop) the physiological data value corresponding to the quantitative data value at that point. This correlation may be performed in any suitable manner. For example, in some embodiments the acquired ultrasound data and acquired physiological monitoring data are time-stamped to allow correlation of the different received data. Using the time-stamps, a correlation between data acquired by different devices, for example, the ultrasound probe and the physiological monitoring device may be performed.

Thereafter, the correlated physiological data is displayed in combination with the quantitative data. For example, one or more plots of physiological monitoring data are displayed at 62, which are time aligned with the plots 88 of the color flow quantitative data in the graph 96 illustrated in FIG. 3. For example, a physiological trace 100, such as an ECG trace may be time aligned (on the same time scale) within the graph 96, such that the data in the trace 100 corresponds in time to the data of the plots 88. Additional physiological data 102, illustrated as a heart rate data also may be displayed.

Thus, acquired data including correlated quantitative data and physiological data from an ultrasound loop, for example, a plurality of heartbeats of a patient, may be displayed in time aligned manner, such as on a single graph 96. It should be noted that the quantitative data may be any type of quantitative data or parameter. For example, although color flow and power Doppler quantitative data may be displayed, other types of quantitative data may be displayed, such as grayscale or volume data (namely three-dimensional data instead of two-dimensional data). For example, the grayscale data may be the mean intensity of the grayscale values from the B-mode image for the ROI versus other regions (e.g., relative brightness of plot versus lumen being imaged). The physiological data may be used to gate and quantify the acquired planar or volume data, for example, to tie the data with the cardiac cycle (e.g., systole or diastole).

Referring to FIG. 2, additional or optional steps may be performed. In particular, a determination may be at 64 as to whether a user input has been received to change or modify any of the displayed data, particularly the displayed quantitative data, based on a review of the displayed physiological monitoring data. For example, a user may decide to scale the graph 96, in particular the plots 88 of the quantitative data, based on the data values for the physiological monitoring data. As another example, a user may select one or more portions of the plots 88 of the quantitative data to include or exclude based on the values of the physiological monitoring data.

If no user inputs are received, then the data continues to be displayed at 66. If user inputs are received to change the displayed data, such as the format of the data or the data portions to be displayed, the data, plots, graph, etc. are updated at 68 based on the user changes.

It should be noted that the user may make selection or changes using a user input device, such as a mouse. The display 80 as shown in FIG. 3 may also include additional user selectable members (e.g., icons) allowing a user to initiate or perform additional or alternative operations. For example, ROI type selection members 104 may be provided to allow a user to identify the ROI 86 on the ultrasound image 82 using a free-hand trace or a predefined shape (e.g., oval, ellipsoid or circle), which is sizeable such that a user can change the height, width and tilt angle of the shape. Additionally, different display formats, such as a short or long display form of data may be selected using the data form selection members 106. For example, the long form display may display more quantitative data or values than the short form display, such as providing maximum values (e.g., maximum values for mean gray scale, color pixel ratio, positive and negative velocity flow, etc.) and the frames in which the maximum values occur.

Additionally, selection members 108 allow a user to select different operations, such as a caliper operation to measure the size of a displayed object. Further, other information may be displayed, such as thumbnail images 110, which may correspond to individually acquired frames or cine loops that are saved in the ultrasound system.

Thus, the various embodiments provide different display and operation options for a user. For example, a user may select a value and image frame of interest based on a physiological input and use that data to normalize the data, such as based on a body temperature from different ultrasound exams, a heart rate, a blood pressure, etc. As other examples, an operator of the system may wait for a steady state of the physiological monitoring data before recording ultrasound data. Accordingly, by practicing various embodiments, a user is able to assess a physical or physiological state of the patient using external inputs, such that the only difference in the acquired data, for example, between different exams, is based on a course of treatment.

The various embodiments, including the display of information, may be provided after acquisition based on data stored in memory or while image acquisition is occurring, such as when a view is frozen. The physiological monitoring data is stored with or in combination with the underlying ultrasound data, for example, the color flow data is stored as raw data prior to beamforming. Accordingly, the cine loop may be displayed based on different phases of the physiological data or the ROI, color scale, grayscale levels or other variables or parameters that may be changed after acquisition of the ultrasound data.

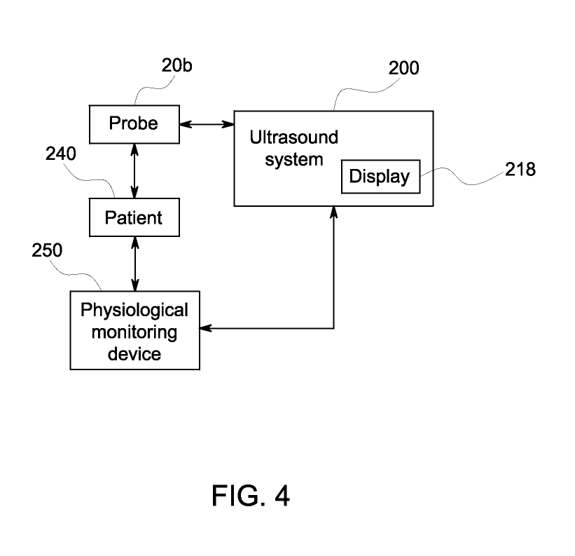

The various embodiments may be implemented in connection with an ultrasound system 200 as illustrated in FIG. 4. The ultrasound system includes a probe 206 for acquiring ultrasound data (e.g., image data) from a patient 240, which may be used to generate the quantitative ultrasound data for display on a display 218. Additionally, a physiological monitoring device 250 is provided for acquiring physiological monitoring data from the patient 240 that is correlated and displayed with the quantitative ultrasound data on the display 218. It should be noted that in various embodiments the probe 206 and/or the physiological monitoring device 250 may or may not form part of the ultrasound system.

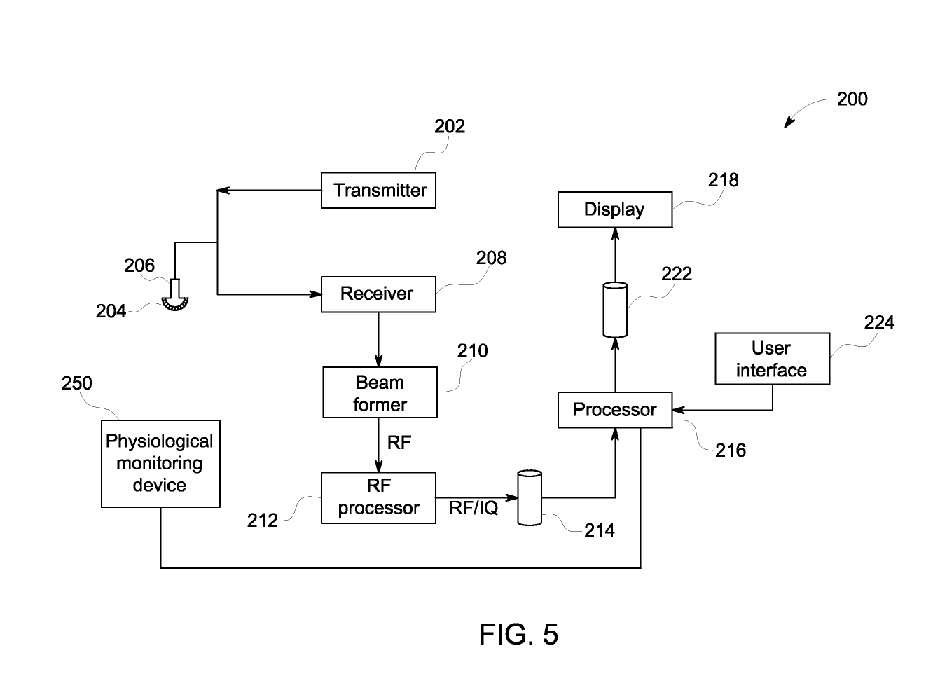

A more detailed block diagram of the ultrasound system 200 is shown in FIG. 5. The ultrasound system 200 is capable of electrical or mechanical steering of a soundbeam (such as in 3D space) and is configurable to acquire information corresponding to a plurality of 2D representations or images of a region of interest (ROI) in a subject or patient, which may be defined or adjusted as described in more detail herein. The ultrasound system 200 is configurable to acquire 2D images in one or more planes of orientation.

The ultrasound system 200 includes a transmitter 202 that, under the guidance of a beamformer 210, drives an array of elements 204 (e.g., piezoelectric elements) within a probe 206 to emit pulsed ultrasonic signals into a body. A variety of geometries may be used. The ultrasonic signals are back-scattered from structures in the body, like blood cells or muscular tissue, to produce echoes that return to the elements 204. The echoes are received by a receiver 208. The received echoes are passed through the beamformer 210, which performs receive beamforming and outputs an RF signal. The RF signal then passes through an RF processor 212. Alternatively, the RF processor 212 may include a complex demodulator (not shown) that demodulates the RF signal to form IQ data pairs representative of the echo signals. The RF or IQ signal data may then be routed directly to a memory 214 for storage.

In the above-described embodiment, the beamformer 210 operates as a transmit and receive beamformer. In an alternative embodiment, the probe 206 includes a 2D array with sub-aperture receive beamforming inside the probe. The beamformer 210 may delay, apodize and sum each electrical signal with other electrical signals received from the probe 206. The summed signals represent echoes from the ultrasound beams or lines. The summed signals are output from the beamformer 210 to an RF processor 212. The RF processor 212 may generate different data types, e.g. B-mode, color Doppler (velocity/power/variance), tissue Doppler (velocity), and Doppler energy, for multiple scan planes or different scanning patterns. For example, the RF processor 212 may generate tissue Doppler data for multi-scan planes. The RF processor 212 gathers the information (e.g. I/Q, B-mode, color Doppler, tissue Doppler, and Doppler energy information) related to multiple data slices and stores the data information, which may include time stamp and orientation/rotation information, in the memory 214. It should be noted that in some embodiments a software beamformer (not shown) may be provided in a back end of the ultrasound system 200 such that the ultrasound data is stored in raw form prior to beamforming.

The ultrasound system 200 also includes a processor 216 to process the acquired ultrasound information (e.g., RF signal data or IQ data pairs) and prepare frames of ultrasound information for display on the display 218. The processor 216 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in memory 214 during a scanning session and then processed and displayed in an off-line operation.

The processor 216 is connected to a user interface 224 (which may include a mouse, keyboard, etc.) that may control operation of the processor 116 as explained below in more detail. The display 218 includes one or more monitors that present patient information, including diagnostic ultrasound images to the user for diagnosis and analysis. One or both of memory 214 and memory 222 may store two-dimensional (2D) or three-dimensional (3D) data sets of the ultrasound data, where such 2D and 3D data sets are accessed to present 2D (and/or 3D images) or physiological monitoring data. The images may be modified and the display settings of the display 218 also manually adjusted using the user interface 224.

It should be noted that although the various embodiments may be described in connection with an ultrasound system and a particular application, the methods and systems are not limited to ultrasound imaging or a particular configuration or application thereof. The various embodiments may be implemented in connection with different types of imaging systems having different configurations or in ultrasound systems having different configurations or in different applications.

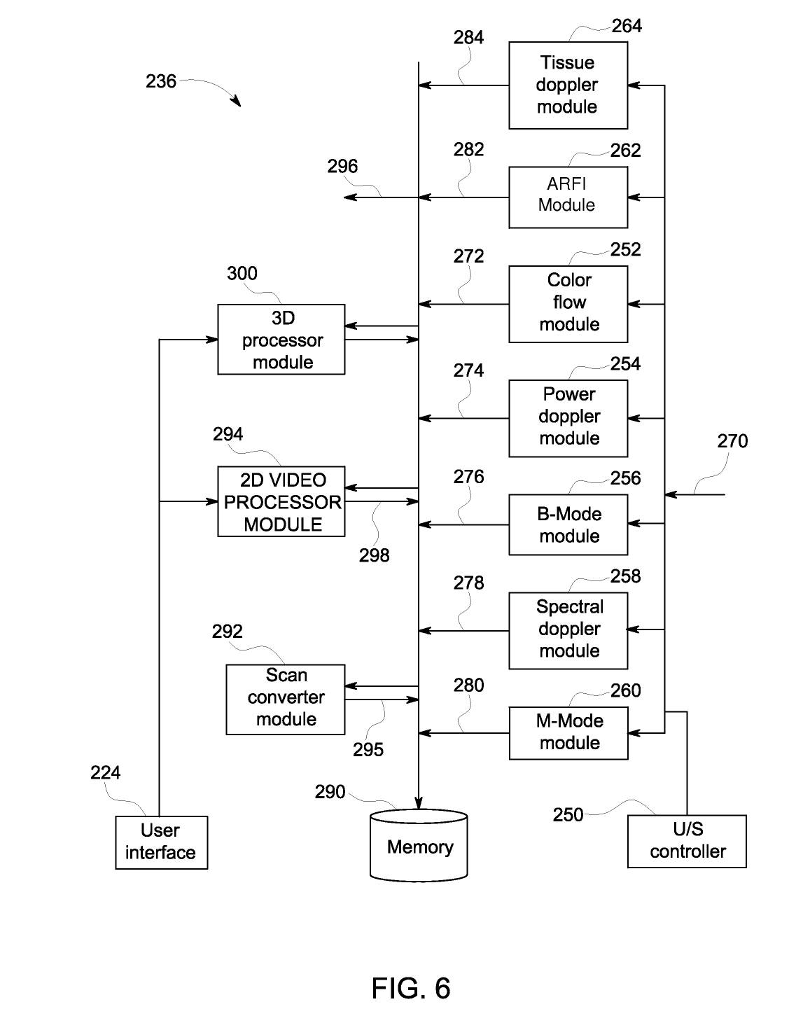

FIG. 6 illustrates an exemplary block diagram of an ultrasound processor module 236, which may be embodied as the processor 216 of FIG. 5 or a portion thereof. The ultrasound processor module 236 is illustrated conceptually as a collection of sub-modules, but may be implemented utilizing any combination of dedicated hardware boards, DSPs, processors, etc. Alternatively, the sub-modules of FIG. 10 may be implemented utilizing an off-the-shelf PC with a single processor or multiple processors, with the functional operations distributed between the processors. As a further option, the sub-modules of FIG. 6 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 6 may be controlled by a local ultrasound controller 242 or by the processor module 236. The sub-modules 252-264 perform mid-processor operations. The ultrasound processor module 236 may receive ultrasound data 270 in one of several forms. In the embodiment of FIG. 6, the received ultrasound data 270 constitutes I,Q data pairs representing the real and imaginary components associated with each data sample. The I,Q data pairs are provided to one or more of a color-flow sub-module 252, a power Doppler sub-module 254, a B-mode sub-module 256, a spectral Doppler sub-module 258 and an M-mode sub-module 260. Optionally, other sub-modules may be included such as an Acoustic Radiation Force Impulse (ARFI) sub-module 262 and a Tissue Doppler (TDE) sub-module 264, among others.

Each of sub-modules 252-264 are configured to process the I,Q data pairs in a corresponding manner to generate color-flow data 272, power Doppler data 274, B-mode data 276, spectral Doppler data 278, M-mode data 280, ARFI data 282, and tissue Doppler data 284, all of which may be stored in a memory 290 (or memory 214 or memory 222 shown in FIG. 5) temporarily before subsequent processing. For example, the B-mode sub-module 256 may generate B-mode data 276 including a plurality of B-mode image planes, such as in a biplane or triplane image acquisition as described in more detail herein.

The data 272-284 may be stored, for example, as sets of vector data values, where each set defines an individual ultrasound image frame. The vector data values are generally organized based on the polar coordinate system.

A scan converter sub-module 292 accesses and obtains from the memory 290 the vector data values associated with an image frame and converts the set of vector data values to Cartesian coordinates to generate an ultrasound image frame 295 formatted for display. The ultrasound image frames 295 generated by the scan converter module 292 may be provided back to the memory 290 for subsequent processing or may be provided to the memory 214 or the memory 222.

Once the scan converter sub-module 292 generates the ultrasound image frames 295 associated with, for example, B-mode image data, and the like, the image frames may be restored in the memory 290 or communicated over a bus 296 to a database (not shown), the memory 214, the memory 222 and/or to other processors.

The scan converted data may be converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 218 (shown in FIG. 5), which may include one or more monitors or windows of the display, to display the image frame. The image displayed in the display 118 is produced from image frames of data in which each datum indicates the intensity or brightness of a respective pixel in the display.

Referring again to FIG. 6, a 2D video processor sub-module 294 combines one or more of the frames generated from the different types of ultrasound information. For example, the 2D video processor sub-module 294 may combine a different image frames by mapping one type of data to a grey map and mapping the other type of data to a color map for video display. In the final displayed image, color pixel data may be superimposed on the grey scale pixel data to form a single multi-mode image frame 298 (e.g., functional image) that is again re-stored in the memory 290 or communicated over the bus 296. Successive frames of images may be stored as a cine loop in the memory 290 or memory 222 (shown in FIG. 5). The cine loop represents a first in, first out circular image buffer to capture image data that is displayed to the user. The user may freeze the cine loop by entering a freeze command at the user interface 224. The user interface 224 may include, for example, a keyboard and mouse and all other input controls associated with inputting information into the ultrasound system 200 (shown in FIG. 5).

A 3D processor sub-module 300 is also controlled by the user interface 124 and accesses the memory 290 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

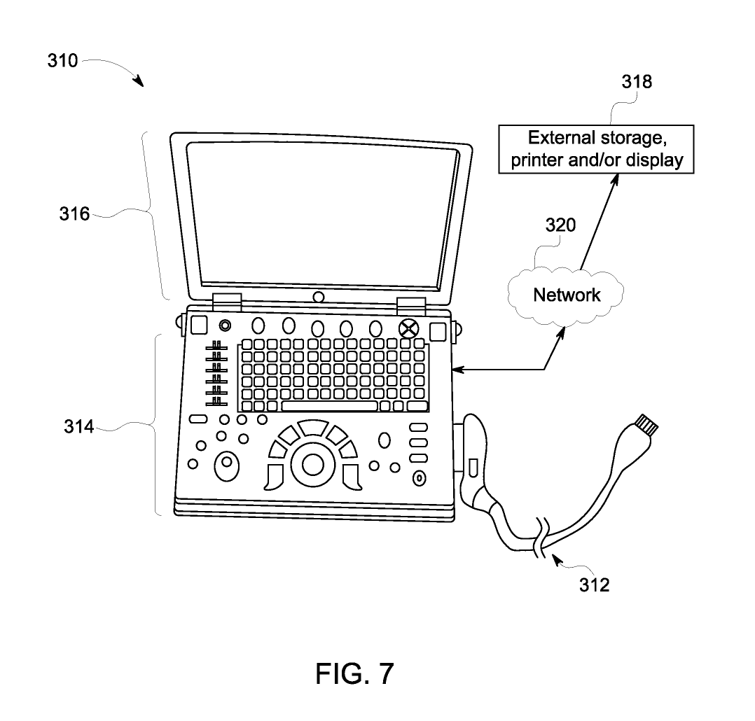

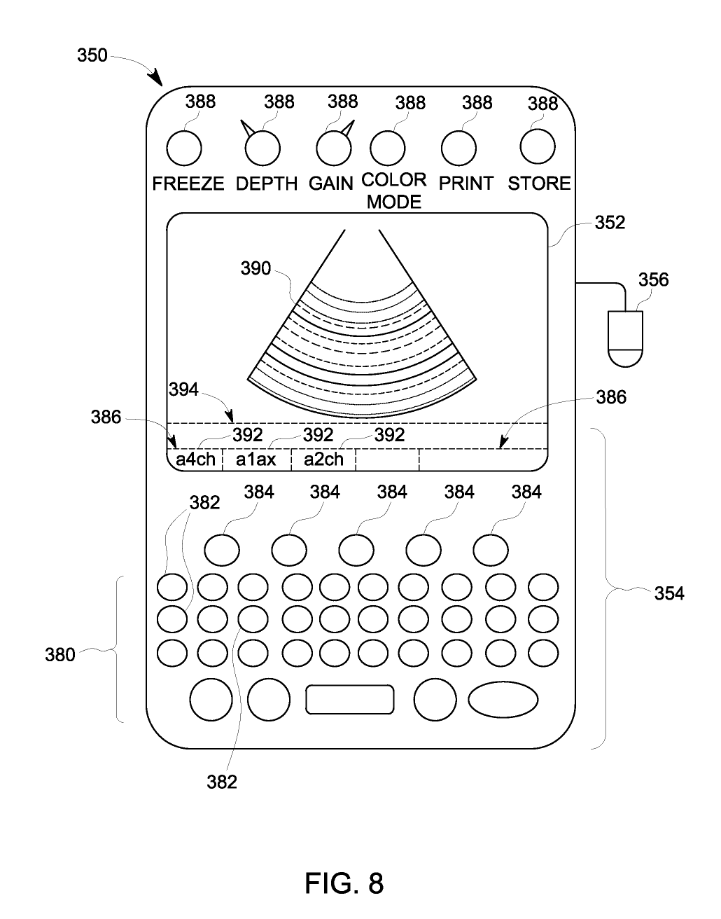

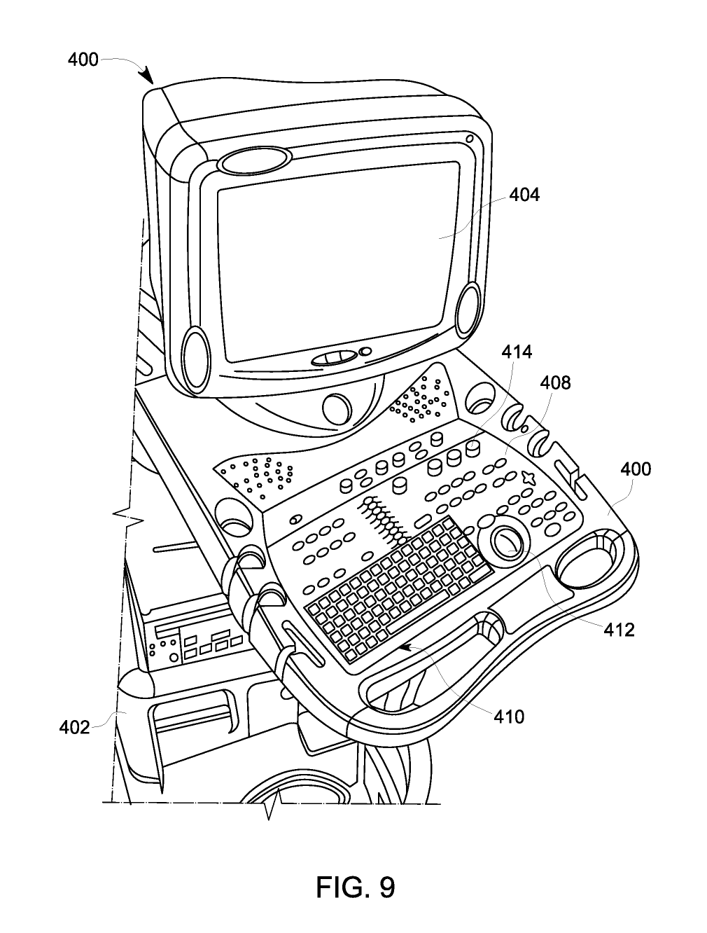

The ultrasound system 200 of FIG. 5 may be embodied in a small-sized system, such as laptop computer or pocket sized system as well as in a larger console-type system. FIGS. 7 and 8 illustrate small-sized systems, while FIG. 9 illustrates a larger system.

FIG. 7 illustrates a 3D-capable miniaturized ultrasound system 310 having a probe 312 that may be configured to acquire 3D ultrasonic data or multi-plane ultrasonic data. For example, the probe 312 may have a 2D array of elements 204 as discussed previously with respect to the probe 206 of FIG. 5. A user interface 314 (that may also include an integrated display 316) is provided to receive commands from an operator. As used herein, “miniaturized” means that the ultrasound system 310 is a handheld or hand-carried device or is configured to be carried in a person's hand, pocket, briefcase-sized case, or backpack. For example, the ultrasound system 310 may be a hand-carried device having a size of a typical laptop computer. The ultrasound system 330 is easily portable by the operator. The integrated display 316 (e.g., an internal display) is configured to display, for example, one or more medical images.

The ultrasonic data may be sent to an external device 318 via a wired or wireless network 320 (or direct connection, for example, via a serial or parallel cable or USB port). In some embodiments, the external device 318 may be a computer or a workstation having a display, or the DVR of the various embodiments. Alternatively, the external device 318 may be a separate external display or a printer capable of receiving image data from the hand carried ultrasound system 310 and of displaying or printing images that may have greater resolution than the integrated display 316.

FIG. 8 illustrates a hand carried or pocket-sized ultrasound imaging system 350 wherein the display 352 and user interface 354 form a single unit. By way of example, the pocket-sized ultrasound imaging system 350 may be a pocket-sized or hand-sized ultrasound system approximately 2 inches wide, approximately 4 inches in length, and approximately 0.5 inches in depth and weighs less than 3 ounces. The pocket-sized ultrasound imaging system 350 generally includes the display 352, user interface 354, which may or may not include a keyboard-type interface and an input/output (I/O) port for connection to a scanning device, for example, an ultrasound probe 356. The display 352 may be, for example, a 320×320 pixel color LCD display (on which a medical image 190 may be displayed). A typewriter-like keyboard 380 of buttons 382 may optionally be included in the user interface 354.

Multi-function controls 384 may each be assigned functions in accordance with the mode of system operation (e.g., displaying different views). Therefore, each of the multi-function controls 384 may be configured to provide a plurality of different actions. Label display areas 386 associated with the multi-function controls 384 may be included as necessary on the display 352. The system 350 may also have additional keys and/or controls 388 for special purpose functions, which may include, but are not limited to “freeze,” “depth control,” “gain control,” “color-mode,” “print,” and “store.”

One or more of the label display areas 386 may include labels 392 to indicate the view being displayed or allow a user to select a different view of the imaged object to display. The selection of different views also may be provided through the associated multi-function control 384. The display 352 may also have a textual display area 394 for displaying information relating to the displayed image view (e.g., a label associated with the displayed image).

It should be noted that the various embodiments may be implemented in connection with miniaturized or small-sized ultrasound systems having different dimensions, weights, and power consumption. For example, the pocket-sized ultrasound imaging system 350 and the miniaturized ultrasound system 310 may provide the same scanning and processing functionality as the system 200 (shown in FIG. 5).

FIG. 9 illustrates an ultrasound imaging system 400 provided on a movable base 402. The portable ultrasound imaging system 400 may also be referred to as a cart-based system. A display 404 and user interface 406 are provided and it should be understood that the display 404 may be separate or separable from the user interface 406. The user interface 406 may optionally be a touchscreen, allowing the operator to select options by touching displayed graphics, icons, and the like.

The user interface 406 also includes control buttons 408 that may be used to control the portable ultrasound imaging system 400 as desired or needed, and/or as typically provided. The user interface 406 provides multiple interface options that the user may physically manipulate to interact with ultrasound data and other data that may be displayed, as well as to input information and set and change scanning parameters and viewing angles, etc. For example, a keyboard 410, trackball 412 and/or multi-function controls 414 may be provided.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, the embodiments are by no means limiting and are exemplary embodiments. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A method for displaying ultrasound data, the method comprising:
acquiring ultrasound image data and physiological monitoring data during an ultrasound imaging scan;
generating quantitative ultrasound data from the acquired ultrasound image data;
correlating the quantitative ultrasound data with the physiological monitoring data; and
displaying the correlated quantitative ultrasound data and physiological monitoring data time aligned on a display.

2. A method in accordance with claim 1 further comprising displaying the quantitative ultrasound data and the physiological monitoring data on a time aligned graph.

3. A method in accordance with claim 1 further comprising displaying an ultrasound image frame generated from the acquired ultrasound image data and receiving a user input defining at least one region of interest (ROI) in the displayed ultrasound image, wherein the correlated quantitative ultrasound data corresponds to the at least one ROI.

4. A method in accordance with claim 3 further comprising associating on the display the ROI with the displayed corresponding correlated quantitative ultrasound data and physiological monitoring data.

5. A method in accordance with claim 1 wherein the quantitative ultrasound data comprises color flow ultrasound data and further comprising displaying at least one plot on a graph corresponding to the color flow ultrasound data time aligned with a physiological monitoring trace based on the physiological monitoring data.

6. A method in accordance with claim 1 wherein the physiological monitoring data comprises at least one of electrocardiography (ECG), heart rate, pulse-oximetry, temperature, blood pressure or breathing data.

7. A method in accordance with claim 1 further comprising displaying the quantitative ultrasound data and the physiological monitoring data on a time aligned graph and scaling the graphed quantitative ultrasound data based on the graphed physiological monitoring data.

8. A method in accordance with claim 1 further comprising displaying the quantitative ultrasound data and the physiological monitoring data on a time aligned graph and receiving a user input to one of include or exclude on the displayed graph quantitative ultrasound data based on the physiological monitoring data.

9. A method in accordance with claim 1 further comprising triggering acquisition of ultrasound image frames when a value of the physiological monitoring data exceeds a threshold value.

10. A method in accordance with claim 1 wherein the quantitative ultrasound data comprises graphical and numerical data including color flow ultrasound data and color flow ratio value data.

11. A method in accordance with claim 1 wherein the quantitative ultrasound data comprises at least one of color flow, power Doppler or B-mode grayscale ultrasound data.

12. A method in accordance with claim 1 further comprising storing the physiological monitoring data with the quantitative ultrasound data stored as raw data.

13. An ultrasound display comprising:
an ultrasound image corresponding to one frame in an acquired ultrasound data image loop;
a quantitative display portion having a time aligned graph;
at least one plot of quantitative ultrasound data on the time aligned graph; and
at least one physiological monitoring trace on the time aligned graph.

14. An ultrasound display in accordance with claim 13 further comprising a frame indicator line on the graph identifying a time at which the ultrasound image frame was acquired corresponding to a time along the physiological monitoring trace.

15. An ultrasound display in accordance with claim 13 further comprising one or more region of interest (ROI) outlines on the ultrasound image and wherein the one or more ROIs are color coded with the at least one plot of quantitative ultrasound data.

16. An ultrasound display in accordance with claim 13 wherein the physiological monitoring trace comprises one of an electrocardiography (ECG), heart rate, pulse-oximetry, temperature, blood pressure or breathing data trace.

17. An ultrasound display in accordance with claim 13 further comprising quantitative ultrasound values corresponding to the at least one plot.

18. An ultrasound system comprising:
a probe configured to acquire ultrasound image data;
a physiological monitoring device configured to acquire physiological monitoring data corresponding to the acquired ultrasound image data;
a processor configured to correlate the acquired ultrasound image data and the acquired physiological monitoring data; and
a display configured to display quantitative ultrasound data based on the ultrasound image data and the physiological monitoring data time aligned.

19. An ultrasound system in accordance with claim 18 further comprising a memory configured to store raw ultrasound image data with the physiological monitoring data.

20. An ultrasound system in accordance with claim 18 wherein the physiological monitoring device comprises at least one of an electrocardiography (ECG), heart rate, pulse-oximetry, temperature, blood pressure or breathing data monitoring device.
